(19) United States
(12) Patent Application Publication
(10) Pub. No.: US 2007/0299512 A1
(43) Pub. Date: Dec. 27, 2007

(54) IMPLANT HAVING A COATING CONTAINING CHOLESTEROL OR CHOLESTEROL ESTER

(75) Inventors: Ellen Korzuschnik, Neunkirchen am Brand (DE); Alexander Borck, Aurachtal (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/767,878

(22) Filed: Jun. 25, 2007

(30) Foreign Application Priority Data
Jun. 26, 2006 (DE) 10 2006 029 247.2

(51) Int. Cl. A61F 2/06

(52) U.S. Cl. 623/1.46

(57) ABSTRACT

A coated implant and a method of use of cholesterol or a cholesterol ester. The implant has a coating which contains one or more components selected from the group of cholesterol and cholesterol esters.

### PRIORITY CLAIM

This patent application claims priority to German Patent Application No. 10 2006 029 247.2, filed Jun. 26, 2006, the disclosure of which is incorporated herein by reference in its entirety.

### FIELD OF THE INVENTION

The present invention relates to a coated implant and a use of cholesterol or a cholesterol ester.

### BACKGROUND OF THE INVENTION

Implants of greatly varying designs have been a fixed component of medical technology for many decades.

For example, the implantation of stents has been established as one of the most effective therapeutic measures in the treatment of vascular diseases. Stents have the purpose of assuming a support function in the interior of the body of a patient. Accordingly, stents are implemented as implantable and have a support structure which ensures the support function. Implants made of metallic materials are known. The selection of metals as a material for the support structure of an implant of this type is based, above all, on the mechanical properties of metals.

A large number of metallic stents are known. One of the main areas of application of such stents is permanently widening and keeping open vascular constrictions, in particular constrictions (stenoses) of the coronary vessels. In addition, aneurysm stents are also known, which offer a support function for a damaged vascular wall. Stents of this type typically have a peripheral wall of sufficient supporting force to keep the constricted vessel open to the desired degree. To allow unobstructed blood flow through the stent, the stent is open at both front ends. More complicated embodiments also allow unobstructed blood flow in secondary vessels. The supporting peripheral wall is typically formed by a latticed support structure, which allows the stent to be inserted in a compressed state having a small external diameter up to the narrow point to be treated of the particular vessel and to be expanded there with the aid of a balloon catheter, for example, enough that the vessel has the desired, enlarged internal diameter. Therefore, the stent has the basic requirement that the support structure has a sufficient supporting force in the expanded state to keep the vessel open. To avoid unnecessary vascular damage, it is additionally desirable for the stent to elastically recoil only slightly after the expansion and after removal of the balloon, so that the stent must only be expanded slightly beyond the desired final diameter during expansion. Further criteria which are desirable in regard to a stent comprise uniform surface coverage and a structure which allows a certain flexibility in relation to the longitudinal axis of the stent, for example.

In some cases, a permanent support function by the stent is not necessary; the body tissue may heal itself in the presence of the stent in such a way that a support effect by the stent no longer appears necessary. This has led to the idea of manufacturing stents from bioresorbable material. In regard to a biodegradable metal stent, it is desirable in addition to the above-mentioned requirements if no or only very slight negative physiological effects originate from the degradation products of the metal stent.

In addition to the desired mechanical properties of the stent, as much as possible the stent is to interact with the body tissue at the implantation location in such a way that renewed vascular constrictions, in particular vascular constrictions caused by the stent itself, do not occur. A restenosis (renewed constriction of the vessel) is to be avoided as much as possible. Currently, stents are used in approximately 70% of all percutaneous interventions; however, in 25% of all cases, there is an in-stent restenosis because of an excessive neointimal growth, which is caused by a strong proliferation of the arterial smooth muscle cells and a chronic inflammation reaction. Greatly varying approaches are followed to reduce the restenosis rates, such as intracoronary radioactive irradiation (brachytherapy).

In a further approach for improving the restenosis problem, the stent is coated with a suitable pharmaceutical active substance, either by directly bonding the active substance to the stent surface or embedding the active substance in a carrier matrix applied to the stent surface. Examples include the active substances SIROLIMUS™ and PACLITAXEL™. Directly bonding active substances to the stent surface has not proven to be very practical; the active substance is overwhelmingly provided in a carrier matrix.

For example, synthetic polymers (e.g., polyurethanes, polymethacrylates, polyvinyl alcohols), degradable polymers (e.g., polyhydroxy butyric acid, polylactides), or polymers of purely biological origin (e.g., hyaluronic acid, phosphorylcholine) may be used as the carrier matrix. However, a part of the polymers cause strong inflammation reactions and thus induce the process of restenosis. Cases of subacute thromboses and allergic reactions have been reported, which were suspected to be caused by the polymers used for the carrier matrix. Patients having multiple or severe symptoms (such as, diabetes, complex lesions, small vessels, or long lesions) display an elevated thrombosis rate, in particular.

The extent to which the components of a carrier matrix actually meet the desired criteria for compatibility upon use in vivo may not be predicted sufficiently precisely solely on the basis of literature data. In biodegradable implants, in particular, interaction with adducts and/or reactive components of the main body is additionally to be observed. Merely finding a material suitable for the coating thus requires a high degree of understanding of the basic biological mechanisms, knowledge of the desired material properties in regard to the processing and later use, and also knowledge about the availability and the possible costs which are connected to the use of the material. Finding such a material is very complex and may not be performed in a standardized way, especially because many material properties which may play a role for the intended use are not yet described or predictable and must first be proven in complex experiments.

Accordingly, there is a need for at least further alternative coating materials for implants, in particular stents.

### SUMMARY OF THE INVENTION

A first feature of the present invention provides an implant having a coating containing one or more components selected from the group of cholesterol and cholesterol esters.

Cholesterol (cholest-5-en-3β-ol; cholesterin) is a colorless substance having a melting point of 148.5° C. Cholesterol is practically insoluble in water, is slightly soluble in cold alcohol and more soluble in warm alcohol, and is soluble in ether, benzene, and petroleum ether. As the main representative of the animal sterols, cholesterol is distributed in all organs: in the cerebrum (approximately 10% of the dry substance), in nerve cells, adrenal glands, and skin. The blood contains 0.15-0.25%, and the heart contains 2% cholesterol. In total, the human body contains an average of 0.32% cholesterol, partially free, partially esterified with fatty acids. Approximately 1-2 g of cholesterol is synthesized daily in the body of adult. The main production location is the liver, but cholesterol is also formed in the adrenal cortex, in the skin, colon, testes, and aorta. Cholesterol and its esters are transported in blood in the form of lipoproteins. Cholesterol, which is included in the lipids, is an important component of biomembranes in addition to phospholipids and glycolipids, in particular, the plasma membranes of eukaryotes, whose fluidity it regulates. Cholesterol also plays a role in the organism as a skin protection substance, swelling regulator, nerve insulator, and the like. Pathologically elevated cholesterol levels may arise in the serum (hypercholesterinemia) due to malnutrition, but also due to specific enzyme or receptor defects. This is considered partially responsible for the occurrence of arteriosclerosis, in which cholesterol-rich deposits form on arterial walls. Cholesterol is used as an emulsifier for cosmetic and pharmaceutical preparations, textile products, leather care agents, and the like, a component of hair growth agents, a starting material for vitamin D synthesis and other steroids, and for cholesterol esters, which are important as liquid crystals.

As a monovalent, secondary alcohol, cholesterol is capable of forming esters (cholesterol ester; cholesterin ester; cholesteryl ester), in particular with aliphatic or aromatic carboxylic acids such as oleic acid, palmitic acid, stearic acid, benzoic acid, linoleic acid, or cinnamic acid. In lipid metabolism, cholesterol esters represent a storage and transportation form of cholesterol. Cholesterol is formed extracellularly with catalysis by lecithin; cholesterol acyltransferase (EC 2.3.1.43), and is stored in lipoproteins.

Surprisingly, it has now been shown that cholesterol and/or its esters may be advantageous components of an implant coating, in particular for stents. From the viewpoint of the applicant, the suitability of cholesterol (esters) is all the more surprising because cholesterol is known to play a supporting role in the occurrence of vascular illnesses and cholesterol intercalations are particularly to be found in the vascular walls precisely in the area of dilated lesions. The particular suitability of cholesterol and/or its esters may be because, as a body-identical product or homolog, cholesterol and/or its esters does not cause any rejection reactions when it is released and/or comes into contact with body tissue. The very small quantities of cholesterol (esters) have no or a very slight effect on the lesions, so that the advantages of using the substances greatly predominate. Accordingly, only the known significance of hypercholesterinemia appears in the foreground as an established risk factor of atherogenesis. In the context of coronary interventions, up to this point no association of serum lipids and restenosis after PTCA alone or also stent implantation has been shown.

A special advantage of the use of cholesterol and/or cholesterol esters is that they may act as a carrier matrix for hydrophobic active substances, such as PACLITAXEL™, PIMECROLIMUS™, or SIROLIMUS™, because of their hydrophobic character. The coating thus preferably additionally contains one or more pharmaceutically active substances, in particular hydrophobic pharmaceutically active substances. In addition, active substances which are not naturally hydrophobic may also be used. The active substances particularly comprise substances for treating in-stent restenosis, for treating secondary effects upon stent implantation, and substances which support the course of healing after implantation.

A coating in the meaning of the present disclosure is an at least partial application of the components to the main body of the implant. If the implant is a stent, the main body of the stent comprises the constructional structures which ensure the mechanical properties of the stent for the above-mentioned purposes. The entire surface of the main body of the stent is preferably covered by the coating. According to a further exemplary variation, the coating may be a depression or hole in the implant body which is filled up with the material, in particular in interaction with pharmaceutically active substances.

Implants preferably comprise—in addition to stents—orthopedic implants such as screws and plates, hip joints, heart valves, bone implants, bypasses, electrodes, and defibrillator and pacemaker housings. It is also conceivable to use the implant as a short-term implant, e.g., in the form of a coated catheter, coated guide wire, or coated electrodes. The implants are entirely or partially provided with the coating according to the present invention.

The coating preferably contains cholesterol and/or cholesterol esters as the main components. A main component in the meaning of the present disclosure is a component of the coating whose weight proportion to the total weight of the coating is greatest. In particular, the weight proportion of the main components is at least 50 weight-percent, especially preferably at least 70 weight-percent. For the case in which the coating contains cholesterol and one or more cholesterol esters, the sum of the weight proportions of these components is preferably at least 50 weight-percent, in particular, at least 70 weight-percent.

The coating preferably additionally contains softeners such as linoleic acid or tocopherol, in particular, in combination with cholesterol (not with cholesterol esters). The admixing of linoleic acid increases the malleability of the coating material and makes it easier to process and apply to the implant, in particular the stent. A weight ratio of linoleic acid to cholesterol is preferably in the range from 1:3 to 1:20.

Furthermore, it is preferable for the cholesterol ester to be cholesterol linoleate, i.e., an ester made of cholesterol and linoleic acid. This ester is especially suitable for use in the human body because of its melting point, which is in the range from 38 to 41° C. according to literature specifications, because the gradual softening of the substance at 37° C. body temperature prevents flaking of the coating during the stent expansion, for example, and the coating covers the stent surface uniformly even after the deformation. The latter property is of special significance, in particular, in connection with biodegradable main bodies, because flaws in the coating represent attack points for main body corrosion, with the result that the degradation of the implant may occur in an uncontrolled way. If the coating contains a combination of cholesterol linoleate and cholesterol, a weight ratio of the ester to the alcohol is preferably in the range from 1:3 to 1:20.

In addition, the coating may contain at least one of the following additives:

Lipophilic vitamins (vitamins A, D, E, K)

Further fatty acids besides linoleic acid (oleic, palmitic, stearic, benzoic, cinnamic, linolenic, arachidonic, myristic, arachidic, behenic, palmitoleic, elaidic, vaccenic, icosenic, cetoleic, erucic, or nervonic acid)

Antioxidants (alpha-tocopherol E 307, ascorbic acid E 300, ascorbyl palmitate E 304, butylhydroxytoluene (BHT) E 321, butylhydroxyanisol (BHA), calcium-disodium-EDTA E 385, calcium-L-ascorbate E 302, calcium hydrogen sulfite E 227, calcium sulfite E 226, citric acid E 330, delta-tocopherol E 309, diphosphate E 450, dodecyl gallate, lauryl gallate E 312, gamma-tocopherol E 308, isoascorbic acid E 315, potassium bisulfite E 228, potassium citrate E 332, potassium sulfite E 224, lecithin E 322, lactic acid E 270, sodium-L-ascorbate E 301, sodium-L-ascorbate E 301, sodium bisulfite E 222, sodium citrate E 331, sodium disulfite E 223, sodium isoascorbate E 316, sodium sulfite E 221, octyl gallate E 311, polyphosphate E 452, propyl gallate E 310, sulfur dioxide E 220, tocopherol E 306, triphosphate E 451, tin-II-chloride E 512)

Emulsifiers (ammonium phoshatide E 442, ascorbyl palmitate E 304, calcium phosphate E 341, calcium stearoyl-2-lactylate E 482, citric acid esters of monoglycerides and diglycerides of dietary fatty acids E 472c, diphosphate E 450, potassium phosphate E 340, lecithin E 322, sodium phosphate E 339, sodium stearoyl-2-lactylate E 481, phosphoric acid E 338, polyglycerin polyricinoleate E 476, polyoxyethylene (40) stearate E 431, polyphosphate E 452, polysorbate 20 E 432, polysorbate 40 E 434, polysorbate 60 E 435, polysorbate 65 E 436, polysorbate 80 E 433, propylene glycol alginate E 405, sorbitan monolaurate E 493, sorbitan monooleate E 494, sorbitan monopalmitate E 495, sorbitan monostearate E 491, sorbitan tristearate E 492, stearyl tartrate E 483, triphosphate E 451, sugar glycerides E 474)

Phospholipids

Fluorescent markers

X-ray markers

Contrast agents for magnetic resonance imaging

Pigments

A tocopherol or a tocopherol derivative is preferably admixed as an additive. A weight ratio of cholesterol (ester) to tocopherol (derivative) is preferably in the range from 3:1 to 1:1.

According to a preferred exemplary embodiment, the implant or stent entirely or partially comprises a biocorrodible metallic alloy, in particular, a magnesium alloy. The implant thus has a main body made of the biocorrodible metallic alloy, whose external surface at least regionally carries the coating. Biocorrodible means that the material is gradually degraded, e.g., by hydrolytic or enzymatic processes, after implantation. Alloys of this type are known, for example, from European Patent Application No. 1 419 793 A1, the content of whose disclosure is referred to in regard to the magnesium alloys used. The use of cholesterol and/or cholesterol esters as a coating material for implants made of a biocorrodible metallic alloy, in particular a magnesium alloy, is especially preferable because the coating materials are known to be hydrophobic and, therefore, a coating of the implant inhibits/delays the degradation processes. In other words, the degradation behavior of the implant may be influenced by a hydrophobic coating of this type. For example, by varying the coating thickness in different areas of the implant, the local degradation behavior of the implant may be influenced. In addition to this preferred exemplary embodiment, coatings on permanent metallic or polymer implants and coatings on degradable polymer implants are also conceivable.

The present invention is explained in greater detail in the following on the basis of an exemplary embodiment.

### EXAMPLES

### Example 1

### Coating of a Biodegradable Stent

A main body of the stent to be coated comprised the biodegradable magnesium alloy WE43.

At room temperature, a solution of 0.2 g cholesterol and 0.2 g alpha-tocopherol was prepared in 3 ml cyclohexane. The stent was immersed in the prepared solution, removed again, and dried at room temperature.

Coated stents were implanted in pigs. An explantation was performed after 35 days. Primary histological evaluations showed that the extent of the restenosis was significantly reduced in relation to uncoated stents.

### Example 2

### Stent Coating Using PIMECROLIMUS™

A main body of the stent to be coated comprised the biodegradable magnesium alloy WE43.

At room temperature, a solution of 0.3 g cholesterol, 0.1 g linoleic acid, and 0.1 g PIMECROLIMUS™ was prepared in 12 ml chloroform. The stent was immersed in the prepared solution, removed again, and dried at room temperature.

All patents, applications and publications referred to herein are incorporated by reference in their entirety.

1. An implant having a coating containing one or more components selected from the group consisting of cholesterol and cholesterol esters.

2. The implant of claim 1, wherein the implant has a main body made of a biocorrodible metallic alloy whose external surface at least regionally carries the coating.

3. The implant of claim 2, wherein the biocorrodible metallic alloy is a magnesium alloy.

4. The implant of claim 1, wherein the coating additionally contains linoleic acid.

5. The implant of claim 4, wherein a weight ratio of linoleic acid to cholesterol is in the range from 1:3 to 1:20.

6. The implant of claim 1, wherein the cholesterol ester is cholesterol linoleate.

7. The implant of claim 1, wherein the coating additionally contains one or more pharmaceutically active substances.

8. The implant of claim 1, wherein the implant is a stent.

9. A method of coating a stent, comprising coating an implantable stent with cholesterol or a cholesterol ester.
